(19) United States
(12) Patent Application Publication
(10) Pub. No.: US 2015/0050364 A1
(43) Pub. Date: Feb. 19, 2015

(54) COMPOSITION FOR TREATING OR PREVENTING TINNITUS

(71) Applicant: UNIVERSITY-INDUSTRY COOPERATION GROUP OF KYUNG HEE UNIVERSITY, Yongin-si, Gyeonggi-do (KR)

(72) Inventors: Tong Ho Kang, Seoul (KR); Bin Na Hong, Seongnam-si (KR)

(73) Assignee: UNIVERSITY-INDUSTRY COOPERATION GROUP OF KYUNG HEE UNIVERSITY, Yongin-si, Gyeonggi-do (KR)

(21) Appl. No.: 14/394,421

(22) PCT Filed: Apr. 18, 2013
(86) PCT No.: PCT/KR2013/003292

§ 371 Date: Oct. 14, 2014

(30) Foreign Application Priority Data
Apr. 18, 2012 (KR) 10-2012-0040357

(51) Int. Cl. A61K 36/43; A23L 1/30; A61K 36/804

(52) U.S. Cl. CPC A61K 36/43; A61K 36/804; A23L 1/3002; A23V 2002/00
USPC 424/725

(57) ABSTRACT

The present invention provides a pharmaceutical composition for treating or preventing tinnitus, comprising the extract of Cuscuta japonica Choisy and Rehmannia glutinosa Libschitz var. purpurea MAKINO. In addition, the present invention provides a functional food for ameliorating or preventing tinnitus, comprising the extract of Cuscuta japonica Choisy and Rehmannia glutinosa Libschitz var. purpurea MAKINO.

### TECHNICAL FIELD

The present invention relates to a composition for treating or preventing tinnitus, comprising the extract of Cuscuta japonica Choisy and Rehmannia glutinosa Libschitz var. purpurea MAKINO.

### BACKGROUND ART

Tinnitus is usually called “an ear ringing” (perception of sound in the absence of an external source of acoustic signal). Tinnitus, a ringing in the ears or an ear ringing is the recognition of sound within the human ear when no corresponding external noise is present. That is, the sound which is not generated outside is heard within the ear.

The tinnitus can appear for a while and disappear, last, or feel like a lasting sound. The tone can be various, and occur in only one ear or in both ears. Statistically, about 15 to 20% of adults experience various tinnitus, and 4% of them suffer from severe tinnitus. In addition, 70 to 80% of the hard of hearing experience tinnitus.

Tinnitus can be classified into objective tinnitus that is heard outside too, and subjective tinnitus that only the patient can hear and that is not heard outside. Further, tinnitus can be classified into peripheral tinnitus and central tinnitus based on the ways perceived by the patients. The peripheral (cochlear) tinnitus is presumed to be derived from the peripheral nervous system and cochlea, and the central tinnitus is estimated to be derived from the auditory cortex.

The objective tinnitus that the doctor can hear the real sound coming from the patient's ear can make a sound like ‘click’ or ‘crackling’ by the convulsion of the muscle around the middle ear. Some people can hear a sound according to the pulse. The pulsatile tinnitus can be generated by the change of the blood flow or the flow of the vein around the ear, or can be subjective symptoms occurring with the increase of interest in the blood flow within the ear.

The subjective tinnitus may have several causes, but it is usually due to otologic disability which results in loss of hearing. The most common cause is noise, and it results from being exposed to excessive or loud noise. However, there can be no clear external factor with sudden loss of hearing. The subjective tinnitus is also known as resulting from the side effects of some medicines like aspirin. Also, the subjective tinnitus can be caused by the side effects of natural hearing dysfunction such as aging and genetic (congenital) hearing dysfunction. The pathophysiologic features of the subjective tinnitus are insufficiently understood, and the exact pathogenesis is unknown so far.

Tinnitus is usually a subjective phenomenon, which cannot be measured through common otologic hearing test methods. This problem was regarded as a factor delaying the development of tinnitus treatment since it acts as a limitation of animal experiments for tinnitus. The tinnitus is clinically evaluated by scoring the subjective pain of the patient through questionnaire. The condition is often clinically rated based on simple evaluation grades from ‘slight’ to ‘severe’ according to practical difficulties such as sleep, meditation, and interference with ordinary behavior. Continuous tinnitus can cause hypersensitivity reaction, tiredness, and clinical depression, etc. In addition, auditory hallucination can be heard.

Most tinnitus patients belong to subjective tinnitus, and the treatment for subjective tinnitus has focused on relaxation of tinnitus through rehabilitation so far. For example, there are a masking method making a sound similar to tinnitus with larger volume than tinnitus from the outside using an instrument like a hearing aid so that tinnitus cannot be heard, and a retraining therapy treating tinnitus without hearing loss by making consistently smaller sound than real tinnitus over a wide frequency. However, there is no tinnitus treatment approved by the US Food and Drug Administration (FDA) so far.

### DISCLOSURE

### Technical Problem

The present inventors discovered that the extract of Cuscuta japonica Choisy and Rehmannia glutinosa Libschitz var. purpurea MAKINO can effectively inhibit tinnitus in tinnitus animal models to complete the present invention.

An object of the present invention is, therefore, to provide a pharmaceutical composition for treating or preventing tinnitus, comprising the extract of Cuscuta japonica Choisy and Rehmannia glutinosa Libschitz var. purpurea MAKINO.

Another object of the present invention is to provide a functional food for ameliorating or preventing tinnitus, comprising the extract of Cuscuta japonica Choisy and Rehmannia glutinosa Libschitz var. purpurea MAKINO.

### Technical Solution

The present invention relates to a pharmaceutical composition for treating or preventing tinnitus, comprising the extract of Cuscuta japonica Choisy and Rehmannia glutinosa Libschitz var. purpurea MAKINO.

Cuscuta japonica Choisy is the seed in egg-shaped fruit of annular plant Cuscuta japonica Choisy belonging to Convolvulaceae. Cuscuta japonica Choisy is known as a medicine protecting liver and kidney, brightening eyes, and strengthening stamina and kidney function.

In one embodiment of the present invention, Cuscuta japonica Choisy comprises Cuscuta chinensis Lam., Cuscuta pentagona Engelm., Cuscuta japonica Choisy, Cuscuta australis R.Br., etc.

Rehmannia glutinosa Libschitz var. purpurea MAKINO is perennial plant belonging to Scrophulariaceae. The raw root is referred to as raw Rehmannia glutinosa Libschitz var. purpurea MAKINO, the dried product is called as dried Rehmannia glutinosa Libschitz var. purpurea MAKINO, and the steamed and dried product is referred to as steamed Rehmannia glutinosa Libschitz var. purpurea MAKINO. Steamed Rehmannia glutinosa Libschitz var. purpurea MAKINO can be used as a hematic and for menstrual irregularity, feeble constitution, stunting in children, dementia, premature ejaculation and erectile dysfunction. Raw Rehmannia glutinosa Libschitz var. purpurea MAKINO can be used for feeble constitution, hematemesis, nosebleed, uterine hemorrhage, menstrual irregularity and constipation. Dried Rehmannia glutinosa Libschitz var. purpurea MAKINO can have effect on thirst occurring after fever and a disease symptomized by thirst due to heat of internal organs, and stop hematemesis and nosebleed.

In one embodiment of the present invention, Rehmannia glutinosa Libschitz var. purpurea MAKINO comprises raw Rehmannia glutinosa Libschitz var. purpurea MAKINO, dried Rehmannia glutinosa Libschitz var. purpurea MAKINO and steamed Rehmannia glutinosa Libschitz var. purpurea MAKINO, of which steamed Rehmannia glutinosa Libschitz var. purpurea MAKINO is preferably used.

In one embodiment of the present invention, the extract of Cuscuta japonica Choisy and Rehmannia glutinosa Libschitz var. purpurea MAKINO represents not only the extract prepared by extracting Cuscuta japonica Choisy and Rehmannia glutinosa Libschitz var. purpurea MAKINO together (the mixed extract of Cuscuta japonica Choisy and Rehmannia glutinosa Libschitz var. purpurea MAKINO) but also the mixture of extracts prepared by extracting them respectively (the mixture of Cuscuta japonica Choisy extract and Rehmannia glutinosa Libschitz var. purpurea MAKINO extract).

In one embodiment of the present invention, the extract of Cuscuta japonica Choisy and Rehmannia glutinosa Libschitz var. purpurea MAKINO preferably comprises about 1.8:1 to 9:1 ratio of Cuscuta japonica Choisy to Rehmannia glutinosa Libschitz var. purpurea MAKINO.

The extract of Cuscuta japonica Choisy and Rehmannia glutinosa Libschitz var. purpurea MAKINO can be obtained by extraction process comprising the extraction step of Cuscuta japonica Choisy and Rehmannia glutinosa Libschitz var. purpurea MAKINO with the extraction solvent (the first extraction solvent) selected from the group consisting of an organic solvent and the mixed solvent of water and an organic solvent.

The organic solvent can comprise C₁₋₄ alcohol, ethyl acetate, etc., but is not limited thereto.

The C₁₋₄ alcohol means a straight or branched alcohol having 1 to 4 carbon atoms, which includes methanol, ethanol, n-propanol, i-propanol, n-butanol, etc., but is not limited thereto. Among them, ethanol is preferred.

The extract of Cuscuta japonica Choisy and Rehmannia glutinosa Libschitz var. purpurea MAKINO can be also obtained by extracting Cuscuta japonica Choisy and Rehmannia glutinosa Libschitz var. purpurea MAKINO respectively as above, obtaining Cuscuta japonica Choisy extract and Rehmannia glutinosa Libschitz var. purpurea MAKINO extract, and then mixing them.

The above extraction process can be performed with about 1 to 20 w/w of the first extraction solvent, preferably the mixed solvent of water and ethanol, based on the weight of Cuscuta japonica Choisy and Rehmannia glutinosa Libschitz var. purpurea MAKINO. As for the mixed solvent, about 70% aqueous ethanol can be preferably used.

The extraction can be conducted by extraction methods known in the art, including but not limited to maceration, extraction with hot water, ultrasonic extraction, reflux extraction, etc. The person in the art can employ various extraction temperature ranges appropriate to the extraction method, for example, including but not limited to from 20 to 100 ° C. Further, the extraction time can vary depending on the extraction method and can be appropriately selected by the person in the art, for example, including the range of about 1 hour to 10 days for single or multiple times, but is not limited thereto. Preferably, the extraction can be performed 2 or 3 times with the first extraction solvent at room temperature for about 2 days.

The extract obtained by extraction with the first extraction solvent can be filtered or centrifuged by conventional methods to give liquid form wherein impurities are removed, or the extract in liquid form can be further concentrated under reduced pressure and/or dried by conventional methods to give powder form.

In addition, the extraction process can optionally further comprise the selection step to obtain the fraction with high proportion of active ingredient. That is, the extract obtained by extraction with the first extraction solvent is dispersed in water, and then is extracted with the appropriate second extraction solvent, such as water-saturated C₄ alcohol to raise the proportion of active ingredient in the resulting extract.

The extract of Cuscuta japonica Choisy and Rehmannia glutinosa Libschitz var. purpurea MAKINO of the present invention inhibited tinnitus upon evaluating Skinner's behavioral response and avoidance response in tinnitus animal models. Particularly, the extract of Cuscuta japonica Choisy and Rehmannia glutinosa Libschitz var. purpurea MAKINO of the present invention had superior tinnitus inhibitory effect, as compared with Cuscuta japonica Choisy extract or Rehmannia glutinosa Libschitz var. purpurea MAKINO extract (see Experimental Examples 1 to 4). Therefore, the extract of Cuscuta japonica Choisy and Rehmannia glutinosa Libschitz var. purpurea MAKINO of the present invention can be effectively used for a pharmaceutical composition for treating or preventing tinnitus.

In one embodiment of the present invention, the tinnitus can include objective tinnitus and subjective tinnitus. Also, the tinnitus can comprise peripheral tinnitus and central tinnitus. In particular, the tinnitus can include subjective tinnitus occurring due to various causes such as noise, drugs, aging, trauma, and viruses.

The pharmaceutical composition according to the present invention can be administered orally, e.g., ingestion or inhalation; or parenterally, e.g., injection, deposition, implantation or suppositories. The injection can be, for example, intravenous, intradermal, subcutaneous, intramuscular or intraperitoneal. Depending on the route of administration, the pharmaceutical composition of the present invention may be formulated as tablets, capsules, granules, fine subtilae, powders, sublingual tablets, suppositories, ointments, injection solutions, emulsions, suspensions, syrups, aerosols, etc. The above various forms of the pharmaceutical composition of the present invention can be prepared in a manner well known in the art using a pharmaceutically acceptable carrier(s) which are usually used for each form. Examples of the pharmaceutically acceptable carriers include excipient, filler, expander, binder, disintegrating agent, lubricant, preservative, antioxidant, isotonic agent, buffer, coating agent, sweetening agent, dissolvent, base, dispersing agent, wetting agent, suspending agent, stabilizer, colorant, etc.

The pharmaceutical composition of the present invention contains about 0.01 to 95 wt % of the extract of Cuscuta japonica Choisy and Rehmannia glutinosa Libschitz var. purpurea MAKINO depending on the form thereof.

The specific dosage of the pharmaceutical composition of the present invention can be varied with species of mammals including a human-being, body weight, gender, severity of disease, judgment of doctor, etc. It is preferable that 0.01 to 50 mg of the active ingredient is administered per kg of body weight a day for oral use, while 0.01 to 10 mg of the active ingredient is administered per kg of body weight a day for parenteral use. The total daily dosage can be administered once or over several times depending on the severity of disease, judgment of doctor, etc.

Another aspect of the present invention relates to a functional food for ameliorating or preventing tinnitus, comprising the extract of Cuscuta japonica Choisy and Rehmannia glutinosa Libschitz var. purpurea MAKINO.

There is no particular limit to the kinds of the functional food according to the present invention. It can be oral preparation such as powders, granules, tablets, capsules, suspensions, emulsions, syrups, etc., or ordinary food such as candies, snacks, gums, ice creams, noodles, breads, drinks, etc. to which the active ingredient is added.

The functional food according to the present invention can be prepared in a manner well known in the art using a sitologically acceptable carrier(s) such as filler, extender, binder, wetting agent, disintegrator, sweetening agent, flavoring agent, preservative, surfactant, lubricant, excipient, etc. depending on the form thereof.

The functional food of the present invention contains about 0.01 to 95 wt % of the extract of Cuscuta japonica Choisy and Rehmannia glutinosa Libschitz var. purpurea MAKINO depending on the form thereof. Since the functional food of the present invention is free from toxicity and side effects, it can be safely used for a long period of time for preventive purpose.

### Advantageous Effects

The extract of Cuscuta japonica Choisy and Rehmannia glutinosa Libschitz var. purpurea MAKINO according to the present invention can inhibit tinnitus in tinnitus animal models and, in particular, have significantly superior tinnitus inhibitory effect, as compared with Cuscuta japonica Choisy extract or Rehmannia glutinosa Libschitz var. purpurea MAKINO extract. Accordingly, the extract of Cuscuta japonica Choisy and Rehmannia glutinosa Libschitz var. purpurea MAKINO can be effectively used for a pharmaceutical composition or a functional food for treating or preventing tinnitus.

### DESCRIPTION OF DRAWINGS

FIG. 1 is a graph showing the true positive ratio and the false positive ratio of Skinner's behavioral response when evaluating the effect of the mixed extract of Cuscuta japonica Choisy and Rehmannia glutinosa Libschitz var. purpurea MAKINO in tinnitus animal models (Normal: no tinnitus occurred, normal group; Control: tinnitus occurred, non-treated control group; TS100: tinnitus occurred, test group treated with the mixed extract of Cuscuta japonica Choisy and Rehmannia glutinosa Libschitz var. purpurea MAKINO).

FIG. 2 is a graph showing the active avoidance ratio and the false avoidance ratio of the avoidance response when evaluating the effect of the mixed extract of Cuscuta japonica Choisy and Rehmannia glutinosa Libschitz var. purpurea MAKINO in tinnitus animal models (Normal: no tinnitus occurred, normal group; Control: tinnitus occurred, non-treated control group; TS100: tinnitus occurred, test group treated with the mixed extract of Cuscuta japonica Choisy and Rehmannia glutinosa Libschitz var. purpurea MAKINO).

FIG. 3 is a graph showing comparative test results of the mixed extract of Cuscuta japonica Choisy and Rehmannia glutinosa Libschitz var. purpurea MAKINO and Cuscuta japonica Choisy extract in tinnitus animal models (Normal: no tinnitus occurred, normal group; Control: tinnitus occurred, non-treated control group; TS100: tinnitus occurred, test group treated with the mixed extract of Cuscuta japonica Choisy and Rehmannia glutinosa Libschitz var. purpurea MAKINO; T100: tinnitus occurred, comparative test group treated with Cuscuta japonica Choisy extract).

FIG. 4 is a graph showing comparative test results of the mixed extract of Cuscuta japonica Choisy and Rehmannia glutinosa Libschitz var. purpurea MAKINO and Rehmannia glutinosa Libschitz var. purpurea MAKINO extract in tinnitus animal models (Normal: no tinnitus occurred, normal group; Control: tinnitus occurred, non-treated control group; TS100: tinnitus occurred, test group treated with the mixed extract of Cuscuta japonica Choisy and Rehmannia glutinosa Libschitz var. purpurea MAKINO; S100: tinnitus occurred, comparative test group treated with Rehmannia glutinosa Libschitz var. purpurea MAKINO extract).

### BEST MODE

The present invention is further illustrated by the following examples, which are not to be construed to limit the scope of the invention.

### EXAMPLE 1

### Preparation of the Mixed Extract of Cuscuta japonica Choisy and Rehmannia glutinosa Libschitz var. Purpurea MAKINO

500 g of the mixture comprising 75 wt % of Cuscuta japonica Choisy and 25 wt % of steamed Rehmannia glutinosa Libschitz var. purpurea MAKINO was added to 10 L of 70 % ethanol and extracted in extraction vessel at 80° C. Supernatant was collected by repeating said procedure 3 times, concentrated with vacuum concentrator (EYELA, N-N) at 45° C., and then lyophilized with lyophilizer (ILSHIN) at the temperature of no more than −40° C. for at least 12 hours to give 97.6 g of the mixed extract of Cuscuta japonica Choisy and Rehmannia glutinosa Libschitz var. purpurea MAKINO.

### EXAMPLE 2

### Preparation of the Mixed Fraction of Cuscuta japonica Choisy and Rehmannia glutinosa Libschitz var. Purpurea MAKINO

50 g of the mixed extract of Cuscuta japonica Choisy and Rehmannia glutinosa Libschitz var. purpurea MAKINO obtained from Example 1 was suspended in 1 L of distilled water and extracted by adding 1 L of water-saturated butanol, and then only the fraction soluble in water-saturated butanol layer was separated from funnel and vacuum concentrated to dry. This procedure was repeated 5 times to give 9.8 g of the fraction.

### COMPARATIVE EXAMPLE 1

### Preparation of Cuscuta japonica Choisy Extract

1000 g of Cuscuta japonica Choisy was added to 10L of 70% ethanol and extracted in extraction vessel at 80 ° C. Supernatant was collected by repeating said procedure 3 times, concentrated with vacuum concentrator (EYELA, N-N) at 45° C., and then lyophilized with lyophilizer (ILSHIN) at the temperature of no more than −40 ° C. for at least 12 hours to give 300 g of Cuscuta japonica Choisy extract.

### COMPARATIVE EXAMPLE 2

### Preparation of Rehmannia glutinosa Libschitz var. Purpurea MAKINO Extract

The same procedure described in Example 1 was conducted except 500 g of steamed Rehmannia glutinosa Libschitz var. purpurea MAKINO was used instead of 500 g of Cuscuta japonica Choisy and steamed Rehmannia glutinosa Libschitz var. purpurea MAKINO to give 104 g of steamed Rehmannia glutinosa Libschitz var. purpurea MAKINO extract.

### Test Animals

As test animals were used 4-week-old female rats, which have normal hearing in an electrophysiological hearing test before training. The normal hearing was less than 30 dB. Before Skinner's behavioral response training, the rats were fasted for 18 hours. After training for 15 minutes, food and water were provided so that the rats could freely eat. Before the avoidance response training, the rats were put into the training and checking box for 10 minutes to let them familiar with the interior space. The rats were not fasted in the avoidance response training. All animals were raised in the environment adjusted to 25±2° C. temperature and 50±10% humidity with the light on every 12 hours.

### EXPERIMENT EXAMPLE 1

### Comparison of True Positive Ratio and False Positive Ratio in Skinner's Behavioral Response When Evaluating the Effect of the Mixed Extract of Cuscuta japonica Choisy and Rehmannia glutinosa Libschitz var. Purpurea MAKINO in Tinnitus Animal Models

When evaluating Skinner's behavioral response with trained animals, the true positive ratio means the ratio of responses pressing the trigger in order to obtain food only when there is a cue tone based on the total responses, and the false positive ratio means the ratio of responses pressing the trigger when there is no cue tone based on the total responses. In case of animals having tinnitus, the false positive ratio pressing the trigger when there is no cue tone tends to increase. Since the animals press the trigger by mistaking the tinnitus sound for the cue tone, the occurrence of tinnitus can be confirmed.

In this experiment, 350 mg/kg of salicylate generally used to induce tinnitus for tinnitus animal models was orally administered to the control group and TS100 group 3 hours before the inspection. The control group was tested without the administration of drugs. After inducing tinnitus, 100 mg/kg of the mixed extract of Cuscuta japonica Choisy and Rehmannia glutinosa Libschitz var. purpurea MAKINO (TS100) prepared in Example 1 was orally administered to the TS100 group an hour and a half before the inspection. The group which did not induce tinnitus was tested as the normal group.

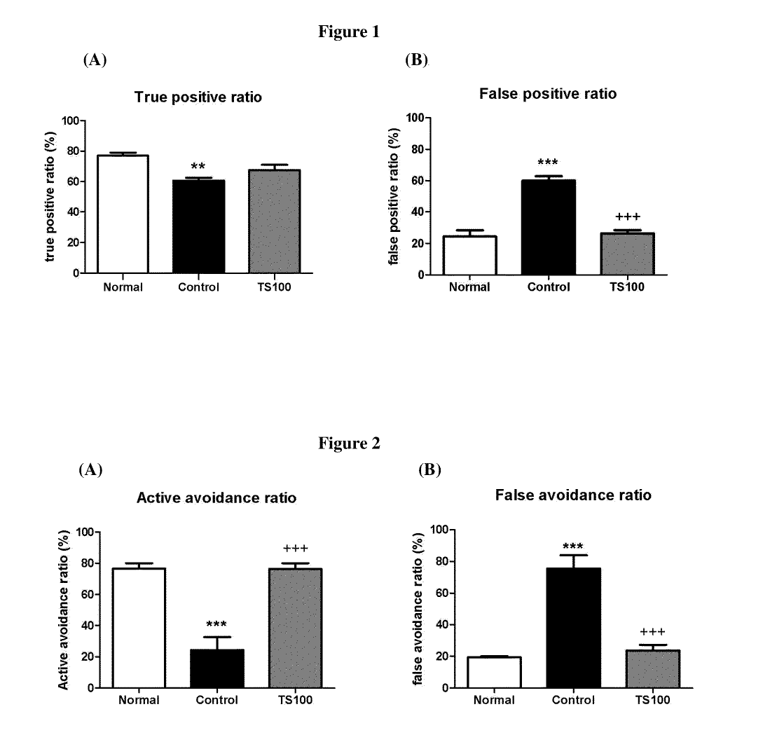

As shown in FIG. 1, the control group significantly lowered the true positive ratio and increased the false positive ratio compared to the normal group to confirm the induction of tinnitus (**p<0.01, ***p<0.001 vs. Normal). The true positive ratios of the control group and the TS100 group were almost similar, but the false positive ratio of the TS100 group was significantly lower compared to the control group, which shows that the tinnitus was suppressed in the TS100 group (+++p<0.001. vs. Control).

### EXPERIMENT EXAMPLE 2

### Comparison of Active Avoidance Ratio and False Avoidance Ratio in the Avoidance Response When Evaluating the Effect of the Mixed Extract of Cuscuta japonica Choisy and Rehmannia glutinosa Libschitz var. Purpurea MAKINO in Tinnitus Animal Models

Experimental Example 1, which uses food when evaluating animal's behavioral response after inducing tinnitus, trains and tests behavioral response while minimizing the stress of animals. However, it is disadvantageous since it takes at least 20 to 30 days to train the animals. To test the behavioral response of animals faster, the avoidance response using an electric stimulus was used. The avoidance response gives a pain by applying an electric stimulus on the floor unless animals run away to the next room within 3 seconds when there is a cue tone. Since the test can be conducted after about 3 day training, the avoidance response can be evaluated much faster than the behavioral response using food. Three days later, most animals succeed in running away to the next room before starting an electric stimulus at least 8 times among 10 times. At this time, the test is started. The avoidance response test gives cue tones 10 times for 10 minutes. The active avoidance ratio means the ratio of the avoidance responses when there is a cue tone based on the total avoidance responses, and the false avoidance ratio means the ratio of the avoidance responses when there is no cue tone based on the total avoidance responses. In case of animals having tinnitus, the active avoidance ratio tends to decrease, and the false avoidance ratio tends to increase.

In this experiment, 350 m of salicylate generally used to induce tinnitus for tinnitus animal models was orally administered to the control group and TS100 group 3 hours before the inspection. The control group was tested without the administration of dings. After inducing tinnitus, 100 mg/kg of the mixed extract of Cuscuta japonica Choisy and Rehmannia glutinosa Libschitz var. purpurea MAKINO (TS100) prepared in Example 1 was orally administered to the TS100 group an hour and a half before the inspection. The group which did not induce tinnitus was tested as the normal group.

As shown in FIG. 2, the control group significantly lowered the active avoidance ratio and increased the false avoidance ratio compared to the normal group to confirm the induction of tinnitus (***p<0.001 vs. Normal). The active avoidance ratio of the TS100 group was significantly higher and the false avoidance ratio of the TS100 group was significantly lower compared to the control group, which shows that the tinnitus was suppressed in the TS100 group (+++p<0.001. vs. Control).

### EXPERIMENTAL EXAMPLE 3

### Comparison of Active Avoidance Ratio and False Avoidance Ratio in the Avoidance Response When Evaluating the Effect of the Mixed Extract of Cuscuta japonica Choisy and Rehmannia glutinosa Libschitz var. Purpurea MAKINO and Cuscuta japonica Choisy Extract in Tinnitus Animal Models

In this experiment, 350 mg/kg of salicylate generally used to induce tinnitus for tinnitus animal models was orally administered to the control group, TS100 group and T100 group 3 hours before the inspection. The control group was tested without the administration of drugs. After inducing tinnitus, 100 mg/kg of the mixed extract of Cuscuta japonica Choisy and Rehmannia glutinosa Libschitz var. purpurea MAKINO (TS100) prepared in Example 1 was orally administered to the TS100 group, and 100 mg/kg of Cuscuta japonica Choisy extract (T100) prepared in Comparative Example 1 was orally administered to the T100 group an hour and a half before the inspection, respectively. The group which did not induce tinnitus was tested as the normal group.

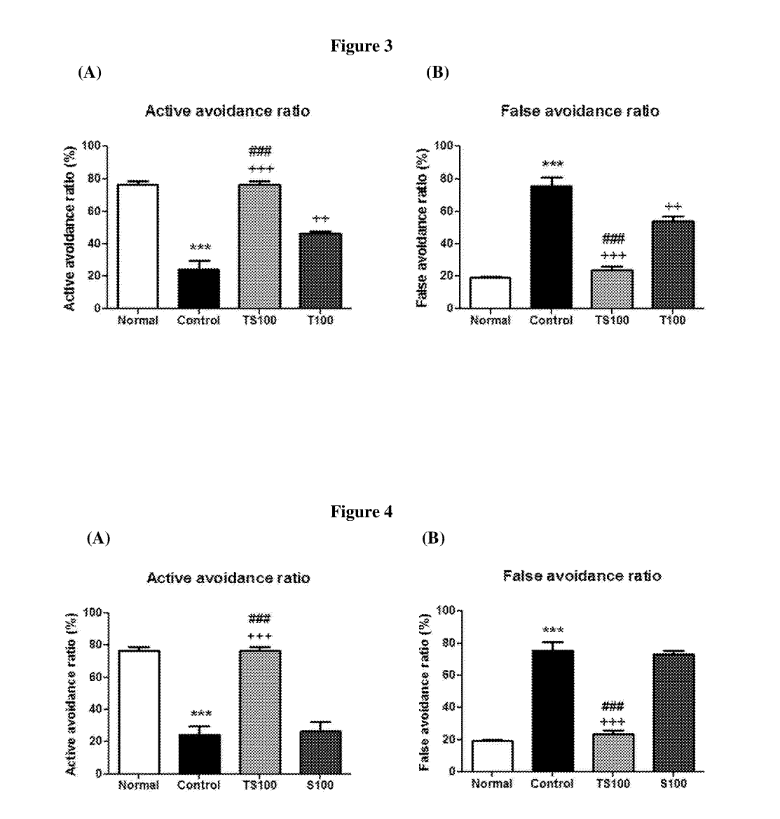

As shown in FIG. 3, the active avoidance ratio of the T100 group was significantly lower and the false avoidance ratio of the T100 group was significantly higher compared to the TS100 group, which shows that the effect of TS100 group was superior to the T100 group (###p<0.001 vs. T100).

### EXPERIMENT EXAMPLE 4

### Comparison of Active Avoidance Ratio and False Avoidance Ratio in the Avoidance Response When Evaluating the Effect of the Mixed Extract of Cuscuta japonica Choisy and Rehmannia glutinosa Libschitz var. Purpurea MAKINO and Rehmannia glutinosa Libschitz var. purpurea MAKINO Extract in Tinnitus Animal Models

In this experiment, 350 m of salicylate generally used to induce tinnitus for tinnitus animal models was orally administered to the control group, TS100 group and S100 group 3 hours before the inspection. The control group was tested without the administration of drugs. After inducing tinnitus, 100 mg/kg of the mixed extract of Cuscuta japonica Choisy and Rehmannia glutinosa Libschitz var. purpurea MAKINO (TS100) prepared in Example 1 was orally administered to the Two group, and 100 mg/kg of Rehmannia glutinosa Libschitz var. purpurea MAKINO extract (S100) prepared in Comparative Example 2 was orally administered to the S100 group an hour and a half before the inspection, respectively. The group which did not induce tinnitus was tested as the normal group.

As shown in FIG. 4, the active avoidance ratio of the S100 group was significantly lower and the false avoidance ratio of the S100 group was significantly higher compared to the TS100 group, which shows that the effect of TS100 group was superior to the S100 group (###p<0.001 vs. S100).

1. A pharmaceutical composition for treating or preventing tinnitus, comprising the extract of Cuscuta japonica Choisy and Rehmannia glutinosa Libschitz var. purpurea MAKINO and a pharmaceutically acceptable carrier.

2. The pharmaceutical composition according to claim 1, wherein Cuscuta japonica Choisy is one or more selected from the group consisting of the seed of Cuscuta chinensis Lam., the seed of Cuscuta pentagona Engelm., the seed of Cuscuta japonica Choisy and the seed of Cuscuta australis R.Br.

3. The pharmaceutical composition according to claim 1, wherein Rehmannia glutinosa Libschitz var. purpurea MAKINO is one or more selected from the group consisting of raw Rehmannia glutinosa Libschitz var. purpurea MAKINO, dried Rehmannia glutinosa Libschitz var. purpurea MAKINO and steamed Rehmannia glutinosa Libschitz var. purpurea MAKINO.

4. The pharmaceutical composition according to claim 1, wherein the extract of Cuscuta japonica Choisy and Rehmannia glutinosa Libschitz var. purpurea MAKINO comprises 1.8:1 to 9:1 ratio of Cuscuta japonica Choisy to Rehmannia glutinosa Libschitz var. purpurea MAKINO.

5. The pharmaceutical composition according to claim 1, wherein the extract of Cuscuta japonica Choisy and Rehmannia glutinosa Libschitz var. purpurea MAKINO is obtained by extraction process comprising the extraction step of Cuscuta japonica Choisy and Rehmannia glutinosa Libschitz var. purpurea MAKINO with the mixed solvent of water and an organic solvent.

6. The pharmaceutical composition according to claim 5, wherein the organic solvent is C₁₋₄ alcohol or ethyl acetate.

7. The pharmaceutical composition according to claim 5, wherein the organic solvent is ethanol.

8. The pharmaceutical composition according to claim 1, wherein the tinnitus is subjective tinnitus.

9. A functional food for ameliorating or preventing tinnitus, comprising the extract of Cuscuta japonica Choisy and Rehmannia glutinosa Libschitz var. purpurea MAKINO.

10. The functional food according to claim 9, wherein Cuscuta japonica Choisy is one or more selected from the group consisting of the seed of Cuscuta chinensis Lam., the seed of Cuscuta pentagona Engelm., the seed of Cuscuta japonica Choisy and the seed of Cuscuta australis R.Br.

11. The functional food according to claim 9, wherein Rehmannia glutinosa Libschitz var. purpurea MAKINO is one or more selected from the group consisting of raw Rehmannia glutinosa Libschitz var. purpurea MAKINO, dried Rehmannia glutinosa Libschitz var. purpurea MAKINO and steamed Rehmannia glutinosa Libschitz var. purpurea MAKINO.

12. The functional food according to claim 9, wherein the extract of Cuscuta japonica Choisy and Rehmannia glutinosa Libschitz var. purpurea MAKINO comprises 1.8:1 to 9:1 ratio of Cuscuta japonica Choisy to Rehmannia glutinosa Libschitz var. purpurea MAKINO.

13. The functional food according to claim 9, wherein the extract of Cuscuta japonica Choisy and Rehmannia glutinosa Libschitz var. purpurea MAKINO is obtained by extraction process comprising the extraction step of Cuscuta japonica Choisy and Rehmannia glutinosa Libschitz var. purpurea MAKINO with the mixed solvent of water and an organic solvent.

14. The functional food according to claim 13, wherein the organic solvent is C₁₋₄ alcohol or ethyl acetate.

15. The functional food according to claim 13, wherein the organic solvent is ethanol.

16. The functional food according to claim 9 wherein the tinnitus is subjective tinnitus.

17. A method for treating tinnitus, comprising administering the composition of claim 1 into a subject in need thereof.
